(19) United States
(12) Patent Application Publication
(10) Pub. No.: US 2009/0208441 A1
(43) Pub. Date: Aug. 20, 2009

(54) PROCESS FOR THE CONTROLLED RADICAL POLYMERIZATION OR COPOLYMERIZATION OF ONE OR MORE MONOMERS IN THE PRESENCE OF AN INITIATOR OF ALKOXYAMINE TYPE

(76) Inventors: Jean-Luc Couturier, Lyon (FR); Manuel Hidalgo, Brignais (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/281,234

(22) PCT Filed: Mar. 2, 2007
(86) PCT No.: PCT/EP2007/052014

§ 371 Date: Dec. 1, 2008

(30) Foreign Application Priority Data
Mar. 2, 2006 (FR) 06 01878

(51) Int. Cl. C08F 2/00; C08F 220/06; C08F 220/18; A61K 8/81

(52) U.S. Cl. 424/78.03; 526/193; 526/317.1; 526/318

(57) ABSTRACT

The invention relates to a process for the polymerization of one or more monomers comprising a stage in which the said monomer or monomers is/are brought into contact, in a medium comprising water, with at least one specific initiator of the alkoxyamine type corresponding, for example, to the following formula:

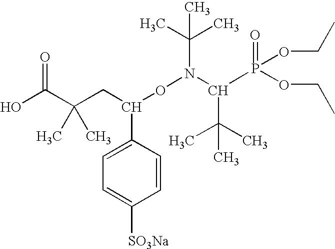

### TECHNICAL FIELD

The present invention relates to a process for the controlled radical polymerization or copolymerization, in particular in aqueous medium, of one or more monomers in the presence of at least one specific initiator of alkoxyamine type.

The general field of the invention is thus that of controlled radical polymerization.

Controlled radical polymerization makes it possible to reduce the reactions in which the growing radical entity is deactivated, in particular the termination stage, which reactions, in conventional radical polymerization, interrupt the growth of the polymer chain in an irreversible and control-free manner.

In order to reduce the probability of the termination reactions, the proposal has been made to transiently and reversibly block the growing radical entity by forming “dormant” entities in the form of bonds with a weak dissociation energy. This thus makes it possible to restart the polymerization and to obtain an alternation between periods of growth of the active radical entities and periods of halting of growth during which these radical entities are dormant. This alternation results in a gradual increase in the average molecular weight as a function of the progression of the polymerization reaction, which takes place, for this reason, in a controlled manner. This control is often reflected by a narrower distribution in molecular weights of the polymer (and thus a lower polydispersity index) than in conventional radical polymerization. This also makes it possible to synthesize block copolymers by restarting the polymerization, from a dormant polymer entity, with a new monomer.

Patent Application FR 2 843 394 describes a controlled radical polymerization process employing alkoxyamines, such as 2-methyl-2-[N-{tert-butyl-N-(diethoxyphosphoryl-2,2-dimethylpropyl)}aminoxy]propionic acid, corresponding to the following general formula:

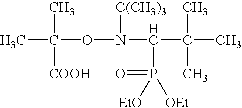

which, when they are used as initiators for a radical polymerization or copolymerization, provide excellent control of the polydispersity while ensuring a good rate of polymerization or of copolymerization.

However, the use of these alkoxyamines may exhibit a number of disadvantages.

Thus, the alkoxyamines described in FR 2 843 394 have to be used in a form neutralized with an excess of strong base in order to be water-soluble. In order to be able to be used for the polymerization or copolymerization of monomers in an aqueous medium and in particular for the polymerization of water-soluble monomers, it is therefore necessary to insert, before the polymerization or copolymerization, a stage of preparation of the alkoxyamine (for example by neutralization with a strong base). Furthermore, depending on the polymerization process employed, it may be necessary, in order to keep these alkoxyamines in solution in an aqueous medium, to make sure that the latter will remain basic throughout a good part of the polymerization, which constitutes a not insignificant limitation as it excludes a number of polymerization processes which take place in an acidic or neutral aqueous medium.

There thus exists a real need for a process for the polymerization of one or more monomers in an aqueous medium which can be employed whatever the pH of the polymerization medium and which does not require a preliminary stage of preparation of the initiator.

The Applicant Company has discovered, surprisingly, that, by using specific initiators of alkoxyamine type, it is possible to carry out a polymerization process exhibiting the abovementioned characteristics.

### ACCOUNT OF THE INVENTION

Thus, the invention relates, according to a first subject-matter, to a process for the polymerization of one or more monomers comprising a stage in which the said monomer or monomers is/are brought into contact, for example in a medium comprising in particular water, with at least one initiator corresponding to the following formula (I):

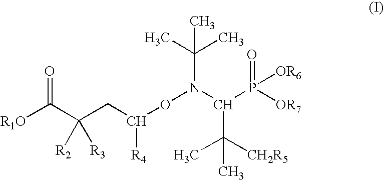

in which:
R₁, represents a hydrogen atom, a linear or branched alkyl group comprising from 1 to 8 carbon atoms, a phenyl group, a metal chosen from alkali metals, alkaline earth metals or transition metals, in particular an alkali metal (Na, Li, K), or also H₄N⁺, Bu₄N⁺ or Bu₃HN⁺, Bu representing an n-butyl group;
R₂ and R₃, which are identical or different, represent a linear or branched alkyl group comprising from 1 to 3 carbon atoms;
R₅ represents a hydrogen atom or an —OCOR₈ group,
R₈ representing a linear or branched alkyl group comprising from 1 to 20 carbon atoms;
R₆ and R₇ independently represent a linear or branched alkyl group comprising from 1 to 3 carbon atoms;
R₄ represents:
an aryl group carrying at least one acid group comprising at least one heteroatom chosen from S and P, it being possible for the said acid group to exist in the form of a salt; or
a heterocyclic group comprising one or more heteratoms chosen from O, N and/or S, the said heterocyclic group optionally carrying at least one acid group comprising at least one heteroatom chosen from S and P or carrying a hydrocarbon group optionally comprising one or more heteroatoms (for example N, S and/or O), the said hydrocarbon group carrying at least one acid group as defined above, it being possible for the said heterocyclic group to exist in the form of a salt; or
a —CO—NR—Y or —CO—O—Y group, with Y representing a hydrocarbon group optionally comprising one or more heteroatoms (for example N, S and/or O) and carrying at least one acid group comprising a heteroatom chosen from S and P or representing a hydrocarbon group optionally comprising one or more heteroatoms (for example N, S and/or O) and comprising at least one heterocyclic group comprising one or more heteroatoms chosen from N, and S, it being possible for the said —CO—NR—Y or —CO—O—Y group optionally to exist in the form of a salt, and R representing a hydrogen atom or an alkyl group preferably comprising from 1 to 24 carbon atoms.

Although the invention is particularly suitable for polymerization in an aqueous medium, it also applies to polymerization in an organic solvent medium.

According to a first alternative, R₄ can be an aryl group comprising, for example, from 5 to 20 carbon atoms (for example a phenyl group), the said aryl group carrying (that is to say substituted by) at least one acid group comprising at least one heteroatom chosen from S and P, it being possible for the said acid group to exist in the form of a salt. Mention may be made, as example of acid group comprising at least one heteroatom chosen from S and P, of a sulphonic, phosphonic, phosphoric or phosphinic group and the salts of these.

According to a second alternative, R₄ can be a heterocyclic group comprising one or more heteroatoms chosen from O, N and/or S, such as a pyrrole, pyridine, indole, thiophene, furan or pyrimidine group, optionally carrying at least one acid group comprising at least one heteroatom chosen from S and P as defined above, or the said heterocyclic group carrying a hydrocarbon group, such as an alkyl group comprising from 1 to 24 carbon atoms, optionally comprising one or more heteroatoms, which hydrocarbon group is substituted one or more times with an acid group as defined above (namely, an acid group comprising at least one heteroatom chosen from S and P).

According to a third alternative, R₄ can represent a —CO—NR—Y or —CO—O—Y group, with Y representing a hydrocarbon group, such as an alkyl group which can comprise from 1 to 24 carbon atoms, optionally comprising one or more heteroatoms and substituted by at least one acid group comprising at least one heteroatom chosen from S and P, such as a sulphonic, phosphonic, phosphoric or phosphinic group. Mention may be made, as example of such group Y, of the —C(CH₃)₂—CH₂SO₃H group. Y can also be a hydrocarbon group, such as an alkyl group comprising from 1 to 24 carbon atoms, comprising optionally one or more heteratoms and carrying at least one heterocyclic group comprising one or more heteroatoms chosen from N, O and S, such as an imidazole, imidazoline, imidazolidone, pyrazole, triazole, tetrazole, thiadiazole or oxadiazole group. The group Y cannot be an unsubstituted alkyl group, in so far as it is necessarily substituted by an acid group or a heterocyclic group as are defined above.

Preference is very particularly given, among initiators of formula (I), to the use of those for which R₄ is an aryl group carrying at least one acid group comprising at least one heteroatom chosen from S and P, it being possible for the said acid group to exist in the form of a salt. In particular, R₄ can advantageously be a phenylene group carrying an —SO₃R₉ group, R₉ representing a hydrogen atom, a metal chosen from alkali metals, alkaline earth metals or transition metals, in particular an alkali metal (Na, Li, K), or also H₄N⁺, Bu₄N⁺ or Bu₃HN⁺, Bu representing an n-butyl group.

A specific initiator in accordance with the invention corresponds to the following formula (II):

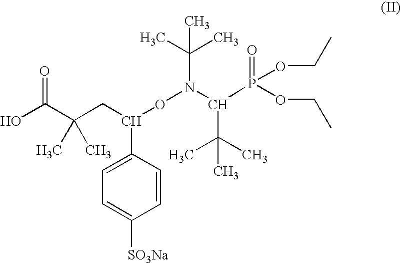

The initiators described above can be obtained by radical addition of the 1,2-type of an olefin comprising a Y functional group to a starting alkoxyamine, according to the following reaction scheme:
a) cleavage of the starting alkoxyamine to give free radicals;

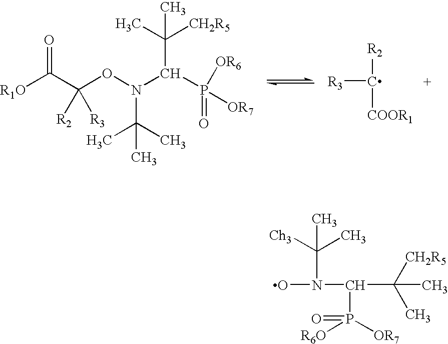

b) radical addition of the 1,2-type of the free radicals formed above to the olefin:

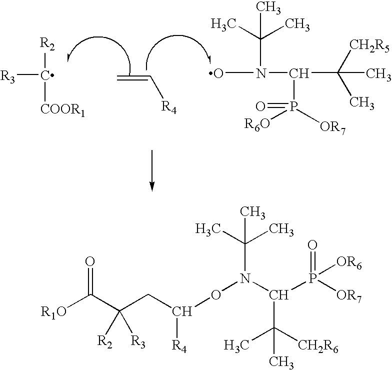

The process is advantageously carried out in a medium comprising water. This medium can be an aqueous or predominantly aqueous solution or a water/organic phase dispersed medium (dispersion, emulsion, miniemulsion, microemulsion, micellar suspension, inverse suspension, inverse emulsion, inverse microemulsion).

As mentioned above, the polymerization process of the invention consists of the polymerization of one or more monomers.

It is understood that the monomer or monomers will in particular be monomers having units, after polymerization, differing from the unit —CH₂—CHR₄— present in the polymerization initiator of formula (I).

In particular, one at least of the monomers can be a water-soluble or water-dispersible monomer.

It is specified that the term “water-soluble monomer” is understood to mean conventionally a monomer which is soluble in water, namely at a content of at least 1% by weight. In other words, this monomer comprises functional groups which are capable of establishing hydrogen bonds with the water molecules which provides it with solubility in water.

The water-soluble monomer or monomers capable of participating in the composition of the polymers or copolymers prepared according to the process of the invention can be chosen from:

water-soluble styrene derivatives, such as sodium styrenesulphonate;

water-soluble acrylic monomers, such as acrylic acid and its salts, methyl acrylate, 2-hydroxyethyl acrylate, 2-methoxyethyl acrylate, methoxy polyethylene glycol acrylates, ethoxy polyethylene glycol acrylates, methoxy polyethylene glycol/polypropylene glycol acrylates and their mixtures, 2-(dimethylamino)ethyl acrylate (ADAME), [2-(acryloyloxy)ethyl]trimethyl-ammonium chloride or sulphate, [2-(acryloyl-oxy)ethyl]dimethylbenzylammonium chloride or sulphate, or alkylene glycol acrylate phosphates;

methacrylic monomers, such as methacrylic acid and its salts, 2-hydroxyethyl methacrylate, 2-ethoxyethyl methacrylate, methoxy polyethylene glycol methacrylates, ethoxy polyethylene glycol methacrylates, methoxy polyethylene glycol/polypropylene glycol methacrylates and their mixtures, 2-(dimethylamino)ethyl methacrylate (MADAME), [2-(methacryloyloxy)ethyl]trimethylammonium chloride or sulphate, [2-(methacryloyloxy)ethyl]dimethylbenzyl-ammonium chloride or sulphate, alkylene glycol methacrylate phosphates, hydroxyethylimidazolidone methacrylate, hydroxyethylimidazolidinone methacrylate or 2-(2-oxo-1-imidazolidinyl)ethyl methacrylate;

acrylamide or substituted acrylamides, N-methylol-acrylamide, acrylamidopropyltrimethylammonium chloride (APTAC), acrylamidomethylpropanesulphonic acid (AMPS) and its salts;

methacrylamide or substituted methacrylamides, 2-methyl-N-[2-(2-oxoimidazolidinyl)ethyl]acrylamide, N-methylolmethacrylamide or methacrylamidopropyl-trimethylammonium chloride (MAPTAC);

itaconic acid, maleic acid and its salts, maleic anhydride, alkyl maleates or hemimaleates, alkoxy or aryloxy polyalkylene glycol maleates or hemimaleates, vinylpyridine or vinylpyrrolidinone; and

a mixture of at least two of the above-mentioned monomers.

The monomers capable of participating in the composition of the polymers or copolymers prepared according to the process of the invention can be hydrophobic monomers chosen from:

vinylaromatic monomers, such as styrene or α-methylstyrene;

diene monomers, such as butadiene or isoprene;

hydrophobic acrylate monomers, such as ethyl acrylate, n-butyl acrylate, ethylhexyl acrylate, phenyl acrylate, methoxy polypropylene glycol acrylates, fluorinated acrylates or silylated acrylates;

methacrylate monomers, such as methyl methacrylate, lauryl methacrylate, cyclohexyl methacrylate, allyl methacrylate, phenyl methacrylate, methoxy polypropylene glycol methacrylates, 2-(tert-butylamino)ethyl methacrylate (MATBAE), fluorinated methacrylates, such as 2,2,2-trifluoroethyl methacrylate, or silylated methacrylates, such as 3-methacryloyloxypropyltrimethylsilane;

acrylonitrile; and

mixtures of these.

The initiator or initiators of formula (I) can be present in a content ranging from 0.005% to 40% by weight, with respect to the total weight of the monomer or monomers employed, and preferably in a content ranging from 0.01% to 10% by weight. The monomer(s) and/or the initiator of alkoxyamine type can optionally be introduced continuously into the polymerization medium.

According to one embodiment of the invention, the contacting stage can be carried out, in addition to the initiator of formula (I), in the presence of an initiator chosen from hydroperoxides, dialkyl peroxides, diacyl peroxides, peroxyesters, peroxydicarbonates, peroxyketals or azo compounds.

Mention may be made, as examples of hydroperoxides, of tert-butyl hydroperoxide, tert-amyl hydroperoxide, cumyl hydroperoxide, 2,5-dimethyl-2,5-di(hydroperoxy)hexane, diisopropylbenzene monohydroperoxide and para-menthane hydroperoxide.

Mention may be made, as examples of dialkyl peroxides, of 2,5-dimethyl-2,5-di(tert-butylperoxy)hex-3-yne, ditert-butyl peroxide, di-tert-amyl peroxide, 1,3-di(tert-butylperoxyisopropyl)benzene, 2,5-dimethyl-2,5-di(tert-butylperoxy)hexyne, 1,1,4,4,7,7-hexamethyl-cyclo-4,7-diperoxynonane or 3,3,6,6,9,9-hexamethyl-cyclo-1,2,4,5-tetraoxanonane.

Mention may be made, as examples of diacyl peroxide, of benzoyl peroxide, lauroyl peroxide, decanoyl peroxide, 3,5,5-trimethylhexanoyl peroxide or acetyl cyclohexylsulphonyl peroxide.

Mention may be made, as examples of peroxyesters, of tert-butyl peroxybenzoate, tert-butyl peroxyacetate, tert-butyl peroxy-3,5,5-trimethyl-hexanoate, tert-amyl peroxy-3,5,5-trimethylhexanoate, 2,5-dimethyl-2,5-di(benzoylperoxy)hexane, OO-tert-butyl O-isopropyl monoperoxycarbonate, OO-tert-butyl O-(2-ethylhexyl)monoperoxycarbonate, OO-tert-amyl O-(2-ethylhexyl)monoperoxycarbonate, tert-butyl peroxyisobutyrate, tert-butyl peroxy-2-ethylhexanoate, tert-amyl peroxy-2-ethylhexanoate, 2,5-dimethyl-2,5-di(2-ethylhexanoylperoxy)hexane, tert-butyl peroxy-neodecanoate, tert-butyl peroxyisononanoate, tert-butyl peroxypivalate, tert-amyl peroxypivalate, α-cumyl peroxyneodecanoate, tert-amyl peroxydecanoate, 3-hydroxy-1,1-dimethylbutyl peroxyneodecanoate and tert-butyl peroxymaleate.

Mention may be made, as examples of peroxydicarbonates, of di(2-ethylhexyl)peroxydicarbonate, dicyclohexyl peroxydicarbonate, di(n-propyl)peroxydicarbonate or di(4-(tert-butyl)cyclohexyl)peroxydicarbonate.

Mention may be made, as examples of peroxyketals, of 1,1-di(tert-butylperoxy)cyclohexane, 1,1-di(tert-butylperoxy)-3,3,5-trimethylcyclohexane, ethyl 3,3-di(tert-butylperoxy)butyrate, ethyl 3,3-di(tert-amylperoxy)butyrate, n-butyl 4,4-di tert-butylperoxy)valerate, 2,2-di(tert-butylperoxy)butane, 1,1-di(tert-amylperoxy)cyclohexane or 2,2-bis[4,4-di(tert-butylperoxy)cyclohexyl]propane.

The contacting stage can also be carried out in the presence of inorganic oxidizing agents, such as sodium persulphate, potassium persulphate or ammonium persulphate, aqueous hydrogen peroxide solution, perchlorates, percarbonates or ferric salts. These oxidizing agents can be used alone or in combination with inorganic or organic reducing agents, such as sodium bisulphite, sodium metabisulphite, potassium bisulphite, potassium metabisulphite, vitamin C, sodium hypophosphite or potassium hypophosphite. These organic or inorganic reducing agents can also be used alone, that is to say in the absence of inorganic oxidizing agents.

The process of the invention is particularly suitable for the preparation of water-soluble (co)polymers by polymerization of one or more water-soluble monomers chosen from:
(meth)acrylic acid and its salts;
(meth)acrylates of amine salts, such as [2-(methacryloyloxy)ethyl]trimethylammonium chloride or sulphate or [2-(methacryloyloxy)ethyl]dimethylbenzyl-ammonium chloride or sulphate;
hydroxyalkyl (meth)acrylates, such as 2-hydroxyethyl methacrylate;
polyethylene glycol, alkoxy or aryloxy polyalkylene glycol (meth)acrylates, such as methoxy polyethylene glycol (meth)acrylates or ethoxy polyethylene glycol (meth)acrylates; and
mixtures of these.

Preferably, the water-soluble monomers used are methacrylic acid, acrylic acid or methoxy polyethylene glycol methacrylates. These copolymers can also comprise a minor fraction of hydrophobic monomer, preferably methyl methacrylate or styrene.

The process is also particularly suitable for the preparation of amphiphilic copolymers, namely copolymers exhibiting both hydrophilic parts and hydrophobic parts. Preferably, the hydrophilic monomers used are methacrylic acid and/or (alkyloxy)polyethylene glycol methacrylates and/or water-soluble cationic monomers, such as [2-(methacryloyl-oxy)ethyl]trimethylammonium chloride, and, preferably, the hydrophobic monomers used are methyl methacrylate and/or styrene and/or 2-(tert-butylamino)ethyl methacrylate (MATBAE).

Finally, the process is also particularly suitable for the preparation of aqueous or organic dispersions of particles according to polymerization techniques well known to a person skilled in the art, such as dispersion or precipitation polymerization, suspension polymerization, miniemulsion polymerization conventional emulsion polymerization, microemulsion polymerization, inverse suspension polymerization, inverse emulsion polymerization, inverse microemulsion polymerization or micellar polymerization, and it is more particularly suitable for the preparation of aqueous dispersions of predominantly hydrophobic particles by the well known techniques of suspension polymerization and emulsion polymerization. In the latter case, the aqueous dispersions of hydrophobic particles obtained are of colloidal size, they scatter light and are known to those skilled in the art under the generic term synthetic “latex”.

The process of the invention is particularly advantageous for preparing fluid latexes, in particular for the purpose of being incorporated in cement, plaster or paint compositions or also cosmetic compositions.

The process of the invention can also comprise a stage of isolation of the polymer or copolymer, for example by precipitation followed by filtration. The isolated polymer or copolymer can be used directly for a given application or can be reintroduced subsequently into a polymerization medium.

The process of the invention can comprise a stage of in situ preparation of the initiator of formula (I), preferably before the contacting stage.

To sum up, the use of initiators of formula (I) in the context of the process has numerous advantages:
they make possible the preparation of polymers in aqueous medium, whatever the pH of the medium and in the presence of acid monomers, such as methacrylic acid, and including in acidic aqueous media, such as those often used in emulsion polymerization;
they make it possible to obtain polymer or copolymer with better control of the polydispersity index than in conventional radical polymerization;
they can make it possible, with an appropriate choice of the monomers to be polymerized and of the polymerization conditions, to obtain living polymer or copolymer, which can be reintroduced in polymerization either so as to increase the molar mass of the said polymer or copolymer or in order to synthesize a block copolymer by introduction of another monomer brought into contact with the living polymer or copolymer synthesized first.

From a structural viewpoint, the process of the invention makes it possible to obtain polymers or copolymers exhibiting at least one reactive end exhibiting the following formula:

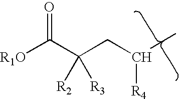

R₁, R₂, R₃ and R₄ being as defined above.

The polymers or copolymers will in particular exhibit a unit of formula —CH₂—CHR₄-resulting from the alkoxyamine present at the end of chains, the other units resulting from the polymerization of the monomers, which units will be different from the said unit —CH₂—CHR₄—.

This polymer or copolymer, exhibiting such a reactive end, can be caused to undergo a chemical conversion at this end by reaction of the latter with appropriate reactants.

The polymers or copolymers synthesized by the process of the invention, by virtue of the nature of its reactive end and of their greater homogeneity in molecular weights, which is reflected by lower polydispersity indices than those obtained with processes and reactants of conventional radical polymerization, are thus novel.

Thus, the invention relates, according to a first subject-matter, to polymers or copolymers capable of being obtained by a process as defined above.

Specific copolymers in accordance with the invention are copolymers comprising repeat units resulting from the polymerization of methacrylic acid, of a methoxy polyethylene glycol methacrylate and of methyl methacrylate.

Another specific copolymer can be a copolymer comprising repeat units resulting from the polymerization of methyl methacrylate and of 2-(tert-butylamino)ethyl methacrylate.

A specific polymer can be a polymer comprising repeat units resulting from the polymerization of methyl methacrylate.

Specific polymers or copolymers in accordance with the invention are amphiphilic polymers or copolymers comprising:
hydrophilic repeat units resulting from the polymerization of methacrylic acid and/or of an (alkyloxy)polyethylene glycol methacrylate and/or of a hydrophilic cationic monomer; and
hydrophobic repeat units resulting from the polymerization of methyl methacrylate and/or of styrene and/or of 2-(tert-butylamino)ethyl methacrylate.

The polymers or copolymers of the invention, in particular when they predominantly comprise water-soluble monomers, can be used in particular as dispersing agents, in particular for pigments or inorganic fillers, in an aqueous medium. They make it possible in particular to give good fluidity to aqueous dispersions of inorganic particles and more particularly to compositions based on hydraulic binders, such as cement and plaster. They also provide a good compromise between the properties of reduction of water and of maintenance of rheology over time. The polymers or copolymers of the invention can also act as dispersants or costabilizers for emulsions of organic products, such as bitumen. They make it possible, in combination with conventional surfactants, to render these emulsions more stable over time.

Specific examples of dispersants or costabilizers in accordance with the invention are copolymers comprising repeat units resulting from the polymerization of methacrylic acid, of a methoxy polyethylene glycol methacrylate and of methyl methacrylate or also a copolymer comprising repeat units resulting from the polymerization of methyl methacrylate and of 2-(tert-butylamino)ethyl methacrylate.

The polymers or copolymers of the invention can in particular also be used as thickening agents, for example in drilling muds, as adhesives, as absorbents or as binders.

Due to their numerous applications, these polymers or copolymers can participate in particular in the composition of numerous compositions, such as:
cosmetic compositions comprising, in addition to the said polymers or copolymers, a cosmetically acceptable medium;
adhesive compositions comprising, in addition to the said polymers or copolymers, additives such as tackifying resins, plasticizers, and the like;
cement compositions;
bitumen compositions;
plaster compositions;
paint compositions.

The invention will now be described with respect to the following examples, given by way of illustration and without implied limitation.

### DETAILED ACCOUNT OF SPECIFIC EMBODIMENTS

The following examples illustrate the preparation of water-soluble copolymers of use in particular as dispersing agents for cement-based preparations.

The dispersing or plasticizing power of the copolymers of the invention can be tested by preparing compositions based on inorganic particles comprising the said copolymer(s).

One of these compositions is cement mortar, obtained by mixing cement, standard sand, water and the aqueous solution or dispersion comprising the dispersing copolymers of the invention optionally comprising antifoaming agents.

Two types of measurements are used in the following examples to characterize the plasticizing power of the copolymers of the invention:
the measurement of the initial spread of a cake of mortar to which a dispersing copolymer has been added;
the measurement of the spread as a function of the time of a cake of mortar to which a dispersing copolymer has been added.

a) Protocol for Measuring the Initial Spread of a Cake of Mortar to which a Dispersing Copolymer of the Invention has been Added

The mortar is prepared in the presence of a dispersing or plasticizing copolymer according to the invention according to standard methods and a hollow truncated cone of well-defined dimensions, known under the name of “Abrams minicone”, is filled with the mortar. The cone has an upper filling opening and a lower emptying opening. The mortar keeps to the inside owing to the fact that the cone is placed on a flat sheet which blocks the lower opening. Immediately after filling, the cone is raised from its support, which has the effect of bringing about the flow of the mortar over the flat sheet, spreading over the latter in the form of a round cake. The more fluid the mortar preparation, the more the mortar will spread and the greater will be the diameter of the cake. The maximum spread diameter of the cake, known to a person skilled in the art under the name of “slump value” or “spread value”, is a fairly reliable measurement of the fluidity of the preparation. This fluidity can vary essentially by adjusting two parameters, which are the Water/Cement ratio (W/C ratio) of the mortar and the content of plasticizing copolymer, expressed as % of dispersing copolymer dry matter with respect to the cement (% SP). The greater the W/C ratio or the higher the % SP, the more fluid the mortar and thus the higher the value given by the measurement of spread diameter.

b) Protocol for Measuring the Spread as a Function of the Time of a Cake of Mortar to which a Dispersing Copolymer of the Invention has been Added

The protocol set out in section a) makes it possible to determine the value of initial spread, that is to say immediately after filling the cone with the mortar for the first time. However, the spread mortar can be recovered and remixed according to a standard procedure and the spread measurement can be repeated at various times starting from the first spread or initial spread. The fluidity of the mortar can thus be monitored over a period generally ranging up to two or three hours. This measurement makes it possible to determine the behaviour of the additivated mortar in terms of change in the fluidity over time. It is often desirable for the fluidity to remain as high as possible for times which are as long as possible, up to the limit of two or three hours.

### Example 1

This example illustrates the preparation of an initiator of following formula (II):

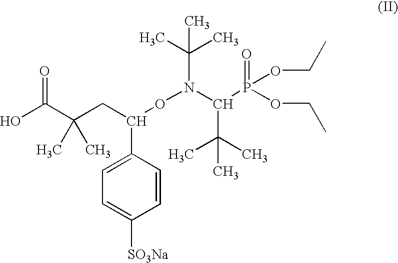

from an alkoxyamine of following formula:

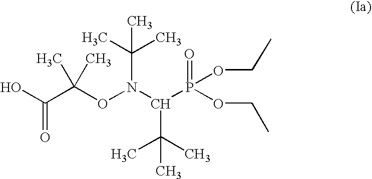

and sodium 4-styrenesulphonate of following formula:

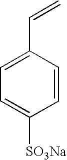

1 litre of ethanol and 0.5 litre of degassed water are introduced into a 2 litre glass rector purged with nitrogen. 54 g of sodium 4-styrenesulphonate (0.262 mol) and 100 g of alkoxyamine of formula (Ia) (0.262 mol) are added. The mixture is heated to 70° C. and left to react for 6 hours with stirring. The product is recovered by evaporating under vacuum at a temperature of 30° C. 179 g of a yellow oil are obtained, which crystallizes in a form of a wax on storing at 4° C.

The product obtained is analysed by ¹H, ¹³C and ³¹P NMR, by negative electrospray mass spectrometry and by Karl Fischer analysis. It comprises 84% by weight of initiator of formula (II), in the form of 2 diastereoisomers in proportions 59/41, and 16% of water.

The characteristics of the alkoxyamine obtained are as follows:

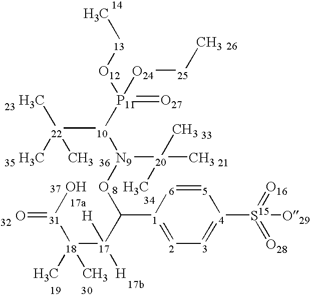

¹H NMR (CDCl₃)

Major isomer

| Atom No. | Chemical shift (in ppm) |
|---|---|
| 2 | 7.45 |
| 3 | 7.7 |
| 5 | 7.7 |
| 6 | 7.45 |
| 7 | 5.1 |
| 19 | 1.13 |
| 30 | 1.13 |
| 10 | 3.4 |
| 17a | 2.3 |
| 17b | 2.5 |
| 21 | 0.8 |
| 23 | 1.10-1.21 |
| 33 | 0.8 |
| 34 | 0.8 |
| 35 | 1.10-1.21 |
| 36 | 1.10-1.21 |
| 25 | 3.2 |
| 13 | 3.65-4.45 |
| 26 | 0.9 |
| 14 | 1.20-1.40 |

Minor isomer

| Atom No. | Chemical shift (in ppm) |
|---|---|
| 7 | 4.9 |
| 6 | 7.3 |
| 2 | 7.3 |
| 5 | 7.7 |
| 3 | 7.7 |
| 10 | 3.32 |
| 23 | 1.10-1.22 |
| 35 | 1.10-1.22 |
| 36 | 1.10-1.22 |
| 26 | 0.9 |
| 13 | 3.65-4.45 |
| 14 | 1.20-1.40 |
| 17 | 2.50-2.75 |
| 19 | 1.1 |
| 21 | 1.19 |
| 25 | 3.65-4.45 |
| 30 | 1.1 |
| 33 | 1.19 |
| 34 | 1.19 |

¹³C NMR (CDCl₃)

Major isomer

| Atom No. | Chemical shift (in ppm) |
|---|---|
| 1 | 141.60-143.40 |
| 2 | 129.50 |
| 3 | 125.00 |
| 4 | 141.60-143.40 |
| 5 | 125.00 |
| 6 | 129.50 |
| 7 | 79.30 |
| 10 | 69.50 |
| 13 | 61.50 |
| 14 | 15.95-16.50 |
| 17 | 43.00 |
| 18 | 39.72 |
| 19 | 22.30 |
| 20 | 61.20 |
| 21 | 28.20 |
| 22 | 35.00 |
| 23 | 30.20 |
| 25 | 58.70 |
| 26 | 15.95-16.50 |
| 30 | 22.30 |
| 31 | 179.80 |
| 33 | 28.20 |
| 34 | 28.20 |
| 35 | 30.20 |
| 36 | 30.20 |

Minor isomer

| Atom No. | Chemical shift (in ppm) |
|---|---|
| 1 | 141.60-143.40 |
| 2 | 128.80 |
| 3 | 124.60 |
| 4 | 141.60-143.40 |
| 5 | 124.60 |
| 6 | 128.80 |
| 7 | 86.60 |
| 10 | 69.40 |
| 13 | 61.50 |
| 14 | 15.95-16.50 |
| 17 | 45.20 |
| 18 | 39.69 |
| 19 | 21.80 |
| 20 | 60.90 |
| 21 | 28.20 |
| 22 | 35.30 |
| 23 | 30.20 |
| 25 | 58.70 |
| 26 | 15.95-16.50 |
| 30 | 21.80 |
| 31 | 179.80 |
| 33 | 28.20 |
| 34 | 28.20 |
| 35 | 30.20 |
| 36 | 30.20 |

³¹P NMR (CDCl₃)

Major isomer: 24 ppm
Minor isomer: 25 ppm

### Example 2

195.6 g of demineralized water are introduced into a 1 litre glass reactor equipped with a variable-speed stirrer motor, inlets for the introduction of reactants, branch pipes for the introduction of inert gases which make it possible to drive off oxygen, such as nitrogen, measurement probes (for example, for measuring temperature), a system for the condensation of vapours with reflux and a jacket which makes it possible to heat/cool the contents of the reactor by virtue of the circulation in the jacket of a heat-exchange fluid, stirring is begun at a moderate speed, the system is heated so as to reach 70° C. in the reactor and degassing is carried out by bubbling nitrogen into the liquid for at least 15 minutes.

Furthermore, the following are prepared in appropriate containers:
1) a mixture comprising:
   259.4 g of Norsocryl N402, consisting of an aqueous solution with a solids content of 58% of the monomer methoxy polyethylene glycol methacrylate (polyethylene glycol side chain with a number-average molecular weight of 2080 g/mol), of methacrylic acid (in a proportion of 2.77%) and of methoxy polyethylene glycol (in a proportion of 5.32%);
   19.6 g of methacrylic acid; and
   26.4 g of methyl methacrylate;
2) 39 g of an aqueous solution (demineralized water) comprising 6% by weight of the initiator (II).

When the temperature of the water in the reactor reaches 70° C., the stirring speed in the reactor is brought to 320 revolutions per minute and the mixture of monomers (1) and the solution of the initiator (II) (2) are then added in parallel to the reactor, using metering pumps, over a period of 2 hours. If necessary, in order to keep the mixture (1) homogeneous, the container comprising it is kept stirred during the two hours of the addition. The solution (2) is homogeneous and does not need to be stirred during the two hours of the addition. During this time, the temperature of the reactor is maintained at least 70° C. At the end of the addition, the temperature is maintained at least 70° C. for at least an additional 3 hours and then the solution is cooled to ambient temperature. The solids content of the aqueous solution of dispersing copolymer thus obtained was measured by gravimetry at 37.1%. The final viscosity of the solution, which has a newtonian behaviour (not dependent on the shear rate), measured with a Brookfield viscosimeter, was 414 mPa·s.

For the measurement of the plasticizing power of the copolymer, a model mortar is prepared starting from 518.5 g of dry cement of Lumbres type (CEM I 42.5R) and 1350 g of standard sand (CEN EN 196-1), 279.99 g of demineralized water and 2.44 g of the aqueous solution of dispersing copolymer, additivated beforehand with 1% by weight with respect to the solids content of an antifoaming agent (Clerol). The amounts employed see to it that the W/C ratio is fixed at 0.54 and the content of dispersant, % SP, at 0.175% with respect to the cement. The preparation of the mortar and the fluidity (spread) measurements are carried out in a room in which the temperature is continuously regulated at 21° C. with a tolerance of plus or minus 2° C.

The mortar is prepared by first mixing the water and the dispersant solution. This mixture constitutes the mixing water, which is subsequently introduced into a mortar mixer. After the addition of the cement, the mixer is started up at 65 revolutions/minute for 30 seconds. After addition of the sand, the mixer is again started up at 65 revolutions/minute for 30 seconds. The mixture is mixed at 125 revolutions/minute for a further 60 seconds. The mortar thus obtained makes it possible to fill the Abrams minicone, the weight and the dimensions of which are as follows:
minimum weight: 4 kg;
diameter of the upper opening: 50 mm;
diameter of the lower opening: 100 mm;
height: 150 mm.

Before filling, the cone is placed on a 50×50 cm PVC sheet with a thickness of 1 cm moistened using a sponge. The cone is filled according to a well-defined procedure consisting in filling in three steps with a third of the height of the cone filled each time and the entire height over a total period of time of 2 minutes. Furthermore, at the end of each third of filling, and in order to reduce the entrainment of air in the mortar, the contents of the cone are settled by rodding them using a metal rod with a length of 30 cm and a diameter of 5 mm. At the end of the two minutes, is the filled cone is lifted up, which causes its contents to spread over the support sheet. 30 seconds after the cone has been lifted up, the spread measurement is taken by measuring the length of two perpendicular diameters of the cake. The mean of these two diameters is the measurement of initial spread of the mortar. For the measurements of fluidity of the mortar as a function of the time, the spread mortar is recovered, replaced in the mixer and left standing and well covered in order to prevent evaporation of water. When the time arrives to carry out a further measurement, mixing is carried out at 125 revolutions/minute for 60 seconds. Subsequently, the cone is filled according to the cited procedure and the spread is again measured as indicated above for the initial spread.

The change in the spread as a function of the time for the mortar prepared with the dispersant in this example is shown in Table I; by way of comparison, this table also shows the performances of a mortar prepared under the same conditions and with the same W/C and % SP values starting from a conventional dispersant (Ecocryl 5930 from Cray Valley).

TABLE 1

| Dispersant | Time (in min) | Spread (in mm) |
|---|---|---|
| Commercial reference | 0 | 340 |
| Ecocryl 5930 | 30 | 310 |
| | 60 | 311 |
| | 90 | 296 |
| | 120 | 275 |
| Dispersant of | 0 | 364 |
| Example 2 | 30 | 363 |
| | 60 | 353 |
| | 90 | 349 |
| | 120 | 340 |

### Example 3

195.6 g of demineralized water are introduced into a 1 litre glass reactor equipped with a variable-speed stirrer motor, inlets for the introduction of reactants, branch pipes for the introduction of inert gases which make it possible to drive off oxygen, such as nitrogen, measurement probes (for example, for measuring temperature), a system for the condensation of vapours with reflux and a jacket which makes it possible to heat/cool the contents of the reactor by virtue of the circulation in the jacket of a heat-exchange fluid, stirring is begun at a moderate speed, the system is heated so as to reach 60° C. in the reactor and degassing is carried out by bubbling nitrogen into the liquid for at least 15 minutes.

Furthermore, the following are prepared in appropriate containers:
1) a mixture comprising:
   259.4 g of Norsocryl N402, consisting of an aqueous solution with a solids content of 58% of the monomer methoxy polyethylene glycol methacrylate (polyethylene glycol side chain with a number-average molecular weight of 2080 g/mol), of methacrylic acid (in a proportion of 2.77%) and of methoxy polyethylene glycol (in a proportion of 5.32%);
   19.6 g of methacrylic acid; and
   26.4 g of methyl methacrylate;
2) 39 g of an aqueous solution (demineralized water) comprising 6% by weight of the initiator (II).

When the temperature of the water in the reactor reaches 60° C., the stirring speed in the reactor is brought to 320 revolutions per minute and the mixture of monomers (1) and the solution of the initiator (II) (2) are then added in parallel to the reactor, using metering pumps, over a period of 2 hours. If necessary, in order to keep the mixture (1) homogeneous, the container comprising it is kept stirred during the two hours of the addition. The solution (2) is homogeneous and does not need to be stirred during the two hours of the addition. During this time, the temperature of the reactor is maintained at least 60° C. At the end of the addition, the temperature is maintained at least 60° C. for at least an additional 3 hours and then the solution is cooled to ambient temperature. The solids content of the aqueous solution of dispersing copolymer thus obtained was measured by gravimetry at 35.8%. The final viscosity of the solution, which has a newtonian behaviour (not dependent on the shear rate), measured with a Brookfield viscosimeter, was 520 mPa·s.

For the measurement of the plasticizing power of the copolymer, the same procedure described in Example 2 is followed and the same experimental conditions are used. The mortar is prepared starting from 518.5 g of dry cement of Lumbres type (CEM I 42.5R) and 1350 g of standard sand (CEN EN 196-1), 279.99 g of demineralized water and 2.53 g of the aqueous solution of dispersing copolymer, additivated beforehand with 1% by weight with respect to the solids content of an antifoaming agent (Clerol).

The change in the spread as a function of the time for the mortar prepared with the dispersant of this example is shown in Table II. By way of comparison, this table also shows the performances of a mortar prepared under the same conditions and with the same values of W/C and % SP starting from a conventional dispersant (Ecocryl 5930 from Cray Valley).

TABLE II

| Dispersant | Time (in min) | Spread (in mm) |
|---|---|---|
| Commercial reference | 0 | 340 |
| Ecocryl 5930 | 30 | 310 |
| | 60 | 311 |
| | 90 | 296 |
| | 120 | 275 |
| Dispersant of | 0 | 371 |
| Example 3 | 30 | 371 |
| | 60 | 369 |
| | 90 | 356 |
| | 120 | 345 |

### Example 4

171.6 g of demineralized water are introduced into a 1 litre glass reactor equipped with a variable-speed stirrer motor, inlets for the introduction of reactants, branch pipes for the introduction of inert gases which make it possible to drive off oxygen, such as nitrogen, measurement probes (for example, for measuring temperature), a system for the condensation of vapours with reflux and a jacket which makes it possible to heat/cool the contents of the reactor by virtue of the circulation in the jacket of a heat-exchange fluid, stirring is begun at a moderate speed, the system is heated so as to reach 70° C. in the reactor and degassing is carried out by bubbling nitrogen into the liquid for at least 15 minutes.

Furthermore, the following are prepared in appropriate containers:
1) a mixture comprising:
   253.8 g of Norsocryl N402, consisting of an aqueous solution with a solids content of 58% of the monomer methoxy polyethylene glycol methacrylate (polyethylene glycol side chain with a number-average molecular weight of 2080 g/mol), of methacrylic acid (in a proportion of 2.77%) and of methoxy polyethylene glycol (in a proportion of 5.32%);
   19.2 g of methacrylic acid; and
   26.2 g of methyl methacrylate;
2) 38.4 g of an aqueous solution (demineralized water) comprising 6% by weight of the initiator (II).

When the temperature of the water in the reactor reaches 70° C., the stirring speed in the reactor is brought to 320 revolutions per minute and the mixture of monomers (1) and the solution of the initiator (II) (2) are then added in parallel to the reactor, using metering pumps, over a period of 2 hours. If necessary, in order to keep the mixture (1) homogeneous, the container comprising it is kept stirred during the two hours of the addition. The solution (2) is homogeneous and does not need to be stirred during the two hours of the addition. During this time, the temperature of the reactor is maintained at least 70° C. At the end of the addition, the temperature is maintained at least 70° C. and 31 g of a 12% by weight solution of sodium metabisulphite (Aldrich) in demineralized water are introduced into the reactor in 20 minutes using a metering pump. The temperature of the reactor is maintained at least 70° C. for at least an additional 3 hours and then the solution is cooled to ambient temperature. The solids content of the aqueous solution of dispersing copolymer thus obtained was measured by gravimetry at 37.8%. The final viscosity of the solution, measured with a Brookfield viscosimeter, was 425 mPa·s. The solution retains an uncoloured appearance as a function of the time.

For the measurement of the plasticizing power of the copolymer, a model mortar is prepared starting from 518.5 g of dry cement of Lumbres type (CEM I 42.5R) and 1350 g of standard sand (CEN EN 196-1), 279.99 g of demineralized water and 2.40 g of the aqueous solution of dispersing copolymer, additivated beforehand with 1% by weight with respect to the solids content of an antifoaming agent (Clerol).

The change in the spread as a function of the time for the mortar prepared with the dispersant of this example is shown in Table III. By way of comparison, this table also shows the performances of a mortar prepared under the same conditions and with the same values of W/C and % SP starting from a conventional dispersant (Ecocryl 5930 from Cray Valley).

TABLE III

| Dispersant | Time (in min) | Spread (in mm) |
|---|---|---|
| Commercial reference | 0 | 339 |
| Ecocryl 5930 | 30 | 319 |
| | 60 | 300 |
| | 90 | 293 |
| | 120 | 272 |
| Dispersant of | 0 | 375 |
| Example 4 | 30 | 375 |
| | 60 | 370 |
| | 90 | 361 |
| | 120 | 354 |

### Example 5

171.6 g of demineralized water are introduced into a 1 litre glass reactor equipped with a variable-speed stirrer motor, inlets for the introduction of reactants, branch pipes for the introduction of inert gases which make it possible to drive off oxygen, such as nitrogen, measurement probes (for example, for measuring temperature), a system for the condensation of vapours with reflux and a jacket which makes it possible to heat/cool the contents of the reactor by virtue of the circulation in the jacket of a heat-exchange fluid, stirring is begun at a moderate speed, the system is heated so as to reach 60° C. in the reactor and degassing is carried out by bubbling nitrogen into the liquid for at least 15 minutes.

Furthermore, the following are prepared in appropriate containers:
1) a mixture comprising:
   253.8 g of Norsocryl N402, consisting of an aqueous solution with a solids content of 58% of the monomer methoxy polyethylene glycol methacrylate (polyethylene glycol side chain with a number-average molecular weight of 2080 g/mol), of methacrylic acid (in a proportion of 2.77%) and of methoxy polyethylene glycol (in a proportion of 5.32%);
   19.2 g of methacrylic acid; and
   26.2 g of methyl methacrylate;
2) 38.4 g of an aqueous solution (demineralized water) comprising 6% by weight of the initiator (II).

When the temperature of the water in the reactor reaches 60° C., the stirring speed in the reactor is brought to 320 revolutions per minute and the mixture of monomers (1) and the solution of the initiator (II) (2) are then added in parallel to the reactor, using metering pumps, over a period of 2 hours. If necessary, in order to keep the mixture (1) homogeneous, the container comprising it is kept stirred during the two hours of the addition. The solution (2) is homogeneous and does not need to be stirred during the two hours of the addition. During this time, the temperature of the reactor is maintained at least 60° C. At the end of the addition, the temperature is maintained at least 60° C. and 31 g of a 12% by weight solution of sodium metabisulphite (Aldrich) in demineralized water are introduced into the reactor in 20 minutes using a metering pump. The temperature of the reactor is maintained at least 60° C. for at least an additional 3 hours and then the solution is cooled to ambient temperature. The solids content of the aqueous solution of dispersing copolymer thus obtained was measured by gravimetry at 37.4%. The final viscosity of the solution, measured with a Brookfield viscosimeter, was 622 mPa·s. The solution retains an uncoloured appearance as a function of the time.

For the measurement of the plasticizing power of the copolymer, a model mortar is prepared starting from 518.5 g of dry cement of Lumbres type (CEM I 42.5R) and 1350 g of standard sand (CEN EN 196-1), 279.99 g of demineralized water and 2.42 g of the aqueous solution of dispersing copolymer, additivated beforehand with 1% by weight with respect to the solids content of an antifoaming agent (Clerol).

The change in the spread as a function of the time for the mortar prepared with the dispersant of this example is shown in Table IV. By way of comparison, this table also shows the performances of a mortar prepared under the same conditions and with the same values of W/C and % SP starting from a conventional dispersant (Ecocryl 5930 from Cray Valley).

TABLE IV

| Dispersant | Time (in min) | Spread (in mm) |
|---|---|---|
| Commercial reference | 0 | 339 |
| Ecocryl 5930 | 30 | 319 |
| | 60 | 300 |
| | 90 | 293 |
| | 120 | 272 |
| Dispersant in | 0 | 383 |
| Example 5 | 30 | 376 |
| | 60 | 375 |
| | 90 | 367 |
| | 120 | 356 |

### Example 6

The following example illustrates the synthesis of a cationic dispersing polymer comprising repeat units resulting from the polymerization of methyl methacrylate and 2-(tert-butylamino)ethyl methacrylate.

358.2 g of demineralized water are introduced into a 1 litre glass reactor equipped with a variable-speed stirrer motor, inlets for the introduction of reactants, branch pipes for the introduction of inert gases which make it possible to drive off oxygen, such as nitrogen, measurement probes (for example, for measuring temperature), a system for the condensation of vapours with reflux and a jacket which makes it possible to heat/cool the contents of the reactor by virtue of the circulation in the jacket of a heat-exchange fluid. Stirring is begun at a moderate speed, the system is heated so as to reach 50° C. in the reactor and degassing is carried out by bubbling nitrogen into the liquid for at least 15 minutes. 0.47 g of polyvinyl alcohol Allotex 72.5 (from Harco) is also introduced into reactor as suspending agent.

The following are furthermore prepared in appropriate containers:
1) a mixture comprising:
   33.3 g of methyl methacrylate; and
   61.6 g of 2-(tert-butylamino)ethyl methacrylate;
2) 46.5 g of an aqueous solution (demineralized water) comprising 10% by weight of the initiator (II).

When the temperature of the water in the reactor reaches 50° C., the stirring speed in the reactor is brought to 500 revolutions per minute (rpm) and the mixture of monomers (1) and the solution of the initiator (II) (2) are then added in parallel to the reactor, using metering pumps, over a period of 60 minutes. During this time, the temperature of the reactor is maintained at least 50° C. At the end of the addition, the temperature is maintained at least 50° C. for at least an additional 2 hours. A “cooking” treatment intended to reduce the residual monomer as much as possible can then be applied, by raising the temperature of the reactor to at least 60° C. for at least an additional 1 hour, and then the suspension is cooled to ambient temperature. The product obtained exists in the form of macroscopic particles (grains or beads) in aqueous suspension. The suspension is removed from the reactor and filtered, so as to separate the hydrophobic polymer (grains) thus obtained from the continuous aqueous phase. The grains, freed from the excess aqueous phase, are subsequently dried in an oven, with or without application of vacuum, care being taken never to exceed 55° C. during the drying in order to prevent the grains from agglomerating. The dried grains can be crushed and sieved, so as to obtain a final product in the form of a homogeneous white powder. This powder, naturally insoluble in water (hydrophobic polymer), can be placed in stirred suspension in water and protonated by the addition of a strong acid, which has the effect of causing it to pass into aqueous solution. Such a solution can be used as dispersing or costabilizing additive, inter alia for the formation of emulsions of organic phases in water (“O/W” or “oil-in-water” emulsions), such as bitumen emulsions.

It should be noted that, in this suspension polymerization, the water-soluble initiator (II) is added instead of the conventional hydrophobic initiator (typically peroxide or azo initiator) soluble in the monomers and that, surprisingly, the result is all the same that of a suspension of grains of hydrophobic polymer in water, as for a conventional suspension.

### Example 7

The following example illustrates the synthesis of a latex of polymethyl methacrylate by emulsion polymerization using an initiator of alkoxyamine type corresponding to the formula (II).

208.01 g of demineralized water are introduced into a 1 litre glass reactor equipped with a variable-speed stirrer motor, inlets for the introduction of reactants, branch pipes for the introduction of inert gases which make it possible to drive off oxygen, such as nitrogen, measurement probes (for example, for measuring temperature), a system for the condensation of vapours with reflux and a jacket which makes it possible to heat/cool the contents of the reactor by virtue of the circulation in the jacket of a heat-exchange fluid. Stirring is begun at a moderate speed, the system is heated so as to reach 70° C. in the reactor and degassing is carried out by bubbling nitrogen into the liquid for at least 15 minutes.

The following are furthermore prepared in appropriate containers:
1) a mixture comprising:
   147.2 g of demineralized water;
   2.86 g of sodium dodecylsulphonate (Rhodacal DS10);
   95.28 g of methyl methacrylate;
2) 46.66 g of an aqueous solution (demineralized water) comprising 4% by weight of the initiator (II).

When the temperature of the water in the reactor reaches 60° C., the stirring speed in the reactor is brought to 300 revolutions per minute (rpm) and half the solution of the initiator (II) (2), i.e. 23.33 g, is introduced into the reactor. When the temperature of the reactor reaches 70° C., 5% by weight, i.e. 12.27 g, of the preemulsion of monomers (1) are added to the reactor. Polymerization is then allowed to take place at least 70° C. for 30 minutes. After this wait of 30 minutes, during which the reaction medium becomes slightly milky due to the formation of colloidal polymer particles, the remainder of the preemulsion of monomers (1) and the remainder of the solution of the initiator (II) (2) are then added in parallel to the reactor, using metering pumps, over a period of time of 120 minutes. During this time, the temperature of the reactor is maintained at least 70° C. At the end of the addition, the temperature is maintained at least 70° C. for at least an additional 2 hours. A “cooking” treatment, intended to reduce the residual monomer as much as possible can be applied by raising the temperature of the reactor to at least 80° C. for at least an additional 1 hour. The product from this emulsion polymerization reaction is a blueish white latex. The latex is cooled at ambient temperature. Its solid content, determined by gravimetry, was 9.1% and its particle size, measured by light scattering (Malvern Lo-C device), a mean diameter of 84 nanometres. The resulting latex comprises a not insignificant amount of residual methyl methacrylate monomer, which can be converted by addition of conventional radical initiators, such as those described above. The residual monomer can also be removed by evaporation, with or without recycling. The final polymethyl methacrylate, freed from the bulk of the residual monomer, can be used in the form of a latex or, after recovery, in the form of a powder in applications appropriate for polymer materials or as additive in preparations, such as adhesives, cosmetics, plastic or inorganic materials, paints or coatings.

1. Process for the polymerization of one or more monomers comprising a stage in which the said monomer or monomers is/are brought into contact with at least one initiator corresponding to the following formula (I): 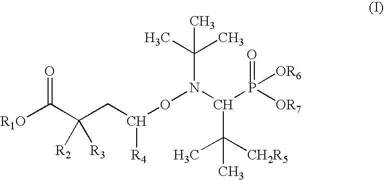
in which:
R₁ represents a hydrogen atom, a linear or branched alkyl group comprising from 1 to 8 carbon atoms, a phenyl group, a metal chosen from alkali metals, alkaline earth metals or transition metals, in particular an alkali metal (Na, Li, K), or also H₄N⁺, Bu₄N⁺ or Bu₃HN⁺, Bu representing an n-butyl group;
R₂ and R₃, which are identical or different, represent a linear or branched alkyl group comprising from 1 to 3 carbon atoms;
R₅ represents a hydrogen atom or an —OCOR₈ group, R₈ representing a linear or branched alkyl group comprising from 1 to 20 carbon atoms;
R₆ and R₇ independently represent a linear or branched alkyl group comprising from 1 to 3 carbon atoms;
R₄ represents:
an aryl group carrying at least one acid group comprising at least one heteroatom chosen from S and P, it being possible for the said acid group to exist in the form of a salt; or
a heterocyclic group comprising one or more heteratoms chosen from O, N and/or S, the said heterocyclic group optionally carrying at least one acid group comprising at least one heteroatom chosen from S and P or carrying a hydrocarbon group optionally comprising one or more heteroatoms, the said hydrocarbon group carrying at least one acid group as defined above, it being possible for the said heterocyclic group to exist in the form of a salt; or
a —CO—NR—Y or —CO—O—Y group, with Y representing a hydrocarbon group optionally comprising one or more heteroatoms and carrying at least one acid group comprising a heteroatom chosen from S and P or representing a hydrocarbon group optionally comprising one or more heteroatoms and comprising at least one heterocyclic group comprising one or more heteroatoms chosen from N, O and S, it being possible for the said —CO—NR—Y or —CO—O—Y group optionally to exist in the form of a salt, and R representing a hydrogen atom or an alkyl group.

2. Process according to claim 1, in which R₄ is an aryl group carrying at least one sulphonic, phosphonic, phosphoric or phosphinic group, it being possible for these groups to exist in the form of salts.

3. Process according to claim 1, in which R₄ is a pyrrole, pyridine, indole, thiophene, furan or pyrimidine group.

4. Process according to claim 1, in which R₄ represents a —CO—NR—Y or —CO—O—Y group with Y representing a hydrocarbon group carrying at least one imidazole, imidazoline, imidazolidone, pyrazole, triazole, tetrazole, thiadiazole or oxadiazole group.

5. Process according to claim 1, in which R₄ is a phenylene group carrying an —SO₃R₉ group, R₉ representing a hydrogen atom, a metal chosen from alkaline metals, alkaline earth metals or transition metals, H₄N⁺, Bu₄N⁺ or Bu₃HN⁺, Bu representing an n-butyl group.

6. Process according to claim 5, in which the initiator corresponds to the following formula (II):

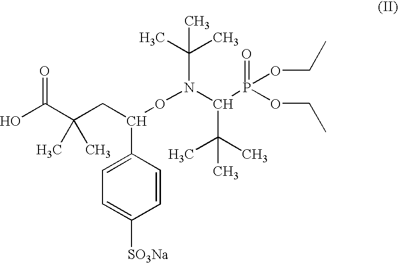

7. Process according to any one of the preceding claims, in which one at least of the monomers is a water-soluble monomer chosen from:
(meth)acrylic acid and its salts;
(meth)acrylates of amine salts;
hydroxyalkyl (meth)acrylates;
polyethylene glycol, alkoxy polyalkylene glycol or aryloxy polyalkylene glycol (meth)acrylates; and
mixtures of these.

8. Process according to claim 1, in which the monomer(s) are chosen from:
vinylaromatic monomers, such as styrene or α-methylstyrene;
diene monomers, such as butadiene or isoprene;
hydrophobic acrylate monomers, such as ethyl acrylate, n-butyl acrylate, ethylhexyl acrylate, phenyl acrylate, methoxy polypropylene glycol acrylates, fluorinated acrylates or silylated acrylates;
methacrylate monomers, such as methyl methacrylate, lauryl methacrylate, cyclohexyl methacrylate, allyl methacrylate, phenyl methacrylate, methoxy polypropylene glycol methacrylates, 2-(tert-butylamino)ethyl methacrylate (MATBAE), fluorinated methacrylates, such as 2,2,2-trifluoroethyl methacrylate, or silylated methacrylates, such as 3-methacryloyloxypropyltrimethylsilane;
acrylonitrile; and
mixtures of these.

9. Process according to claim 1, in which the initiator is present in a content ranging from 0.01 to 10% by weight, with respect to the total weight of the monomer(s).

10. Process according to claim 1, in which the contacting stage is carried out in the presence of one or more initiators chosen from dialkyl peroxides, diacyl peroxides, hydroperoxides, azo compounds and mixtures of these.

11. Process according to claim 1, in which the contacting stage is carried out in the presence of one or more oxidizing agents chosen from the group consisting of sodium persulphate, potassium persulphate, ammonium persulphate, aqueous hydrogen peroxide solution, perchlorates, percarbonates, ferric salts and mixtures of these.

12. Process according to claim 1, in which the contacting stage is carried out in the presence of one or more reducing agents chosen from sodium bisulphite, potassium bisulphite, sodium metabisulphite, potassium metabisulphite, vitamin C, sodium hypophosphite or potassium hypophosphite.

13. Process according to claim 1, comprising a stage of in situ preparation of the initiator of formula (I).

14. Polymer or copolymer capable of being obtained according to a process as defined according to claim 1.

15. Copolymer according to claim 14, comprising repeat units resulting from the polymerization of methacrylic acid, of a methoxy polyethylene glycol methacrylate and of methyl methacrylate or comprising repeat units resulting from the polymerization of methyl methacrylate and of 2-(tert-butylamino)ethyl methacrylate.

16. Copolymer according to claim 14, comprising:
hydrophilic repeat units resulting from the polymerization of methacrylic acid and/or of an (alkyloxy)polyethylene glycol methacrylate and/or of a hydrophilic cationic monomer; and
hydrophobic repeat units resulting from the polymerization of methyl methacrylate and/or of styrene and/or of 2-(tert-butylamino)ethyl methacrylate.

17. Polymer according to claim 14, comprising repeat units resulting from the polymerization of methyl methacrylate.

18. A method for dispersing particles of a particulate composition comprising adding a polymer or copolymer according to claim 14 as as dispersant for particles.

19. Cosmetic composition comprising a polymer or copolymer as defined according to claim 14 and a cosmetically acceptable medium.

20. Cement composition comprising a polymer or copolymer as defined according to claim 14.

21. Bitumen composition comprising a polymer or copolymer as defined according to claim 14.

22. Plaster composition comprising a polymer or copolymer as defined according to claim 14.

23. Paint composition comprising a polymer or copolymer as defined according to claim 14.
